(19) United States
(12) Patent Application Publication
(10) Pub. No.: US 2020/0237810 A1
(43) Pub. Date: Jul. 30, 2020

(54) Treating erectile dysfunction via an herbal compostion at the navel area

(71) Applicants: James Hong Jie Zhou, Richmond, TX (US); Randy Zhou, Richmond, TX (US)

(72) Inventors: James Hong Jie Zhou, Richmond, TX (US); Randy Zhou, Richmond, TX (US)

(73) Assignees: Randy Zhou, Richmond, TX (US); James Hong Jie Zhou, Richmond, TX (US)

(21) Appl. No.: 16/350,893

(22) Filed: Jan. 30, 2019

(51) Int. Cl. A61K 33/04; A61K 9/00; A61K 36/234; A61K 36/23; A61K 36/54; A61K 36/79; A61K 36/46; A61K 36/9068; A61K 47/06; A61K 31/045; A61P 15/10

(52) U.S. Cl. CPC A61K 33/04; A61K 9/0014; A61K 36/234; A61K 36/23; A61K 36/54; A61P 15/10; A61K 36/46; A61K 36/9068; A61K 47/06; A61K 31/045; A61K 36/79

(57) ABSTRACT

The present invention provides a unique combination of herbal ingredients designed to improve human sexual performance and for the treatment of disorders of Male Sexual Dysfunction. The composition is comprised of Sulfur, Cnidium, Petrolatum Jelly, Cumin, Cassia, Schisandra, Eucommiacease, Borneal, Zingiber officinale in amounts effective to produce desired results.

### CROSS REFERENCES TO RELATED APPLICATIONS

This application is based on provisional application Ser. No. 62/709,393 filed Jan. 17, 2018.

### FIELD OF THE INVENTION

This invention relates to a composition for treating male sexual dysfunction.

### BRIEF DESCRIPTION OF THE DRAWINGS

There are no drawing related to this invention.

### BACKGROUND OF THE INVENTION

Erectile dysfunction is the inability to attain or maintain an erect penis that allows satisfactory sexual performance. This problem occurs at some time or other for almost all sexually active males. The problem can occur at any age, although it is most common among males between 55 and 65 years of age. When the problem occurs during more than 25% of sexual encounters, intervention is needed. Currently worldwide, there are “more than 150 million men are affected by erectile dysfunction with an additional 35 million men per year experiencing symptoms for the first time. In the United States, erectile dysfunction is a problem affecting approximately 40 million men enough to warrant intervention. (Elder, 2015 THE MARKET FOR RX-TO-OTC SWITCHES. KALORAMA INFORMATION, (7), 1-26.) The current market solution to erectile dysfunction is the consumption of prescription drugs such as Viagra by Pfizer, or Cialis by Eli Lilly and Co. These drugs have been effective in treating Erectile Dysfunction by providing the patient with short-term results. However, these prescriptions do not provide the patient with a long-term solution in relieving their dysfunction. In addition, the cost of these prescription drugs can vary wildly from $5 per pill to $70 per pill depending on the age and insurance of the patient, which in turn places some consumers at a disadvantage of treating their illness. In addition, the current herbal supplement market for erectile dysfunction has mixed results. Many consumers have tried these products but have not found an effective long-term treatment. Therefore, a new type of treatment is needed by consumers. The composition and method of application is a completely new approach for the treatment of erectile dysfunction. Unlike conventional medication taken orally, the herbal composition relies on vapors emitted from the compound that diffuse across the skin and is directly absorbed into the body through the button area/navel. This special blend of herbs and inorganic compounds produces a pronounced vapor and is activated upon body heat contact.

### BRIEF DESCRIPTION OF THE INVENTION

This invention involves a unique composition of ingredients that is applied as a solid or paste form to the navel area of the patient desiring relief from sexual disfunction or enhancement of sexual function. One important requirement for performance is that the composition be placed on or at the individual's naval. Navel introduction of certain drug compositions has been described in the literature, for example, World J. Gastroenterol 2006 Dec. 28, 12948): 7798-7804.

Whenever possible, naturally occurring herbs and other compounds are chosen for the composition. If the composition of the present invention is applied on or through any other part of the human body its effectiveness is minimized or negated.

### DETAILED DESCRIPTION OF THE INVENTION

The composition used in the present invention contains many components that have been described in the literature for treating male sexual disfunction. Also, some of the components are chosen to produce a gel or paste as is known to those familiar with the art. Through careful evaluation it has been confirmed that the present invention of applying the composition specifically to the navel area gives much more effective performance than applying to other locations of the body.

In one example, the herbal compound can be_comprised of the following materials: Sulfur, Borneol, Cnidium, and Petrolatum Jelly, Cumin, Cassia, Schisandra, Eucommiaceae, Zingiber officinale. In this example, the weight of each ingredient of the final product is listed below.

| Ingredient | Ingredient weight range (Grams) |
|---|---|
| Sulfur | 1 g-30 g |
| Cnidium | 1 g-28 g |
| Petrolatum Jelly | 0.50 g-25 g |
| Cumin | 0.01 g-3 g |
| Cassia | 0.05 g-4 g |
| Schisandra | 1 g-5 g |
| Eucommiacease | 0.01 g-3 g |
| Borneol | 1 g-20 g |
| Zingiber officinale | 0.01 g-6 g |

The composition listed above is for illustration purposes and the invent is not restricted to these ingredients.

Among a group of 70 test subjects, those who applied the composition directly to the navel experience positive sexual performance enhancement. Several subjects applied the composition near but not on the navel that resulted in diminished performance results. Other subjects applied the composition to other area of the body with negligible or no positive results. All patients applied the composition for 14 days. In order to affix the composition to the treated areas an adhesive patch is usually employed. Unexpectedly, sexual performance obtained using the present invention was found to be superior to that obtained through other methods of application or ingredients known to improve performance.

1. The application of a sexual enhancement composition to the navel area of male subjects.

2. The composition described in claim 1 where the ingredients are chosen from those known to the art to give the desired enhancement.

3. The composition described in claim 1 where the composition is a solid, paste, or gel.
